(19) United States
(12) Patent Application Publication
(10) Pub. No.: US 2005/0272037 A1
(43) Pub. Date: Dec. 8, 2005

(54) System and method for solution based multiparameter analysis of analytes

(76) Inventors: Caroline Garey, Cambridge (GB); Jodie Hadley, Ely (GB); Peter Swarbrick, Cambridge (GB)

Correspondence Address: STITES & HARBISON PLLC, 1199 NORTH FAIRFAX STREET, SUITE 900, ALEXANDRIA, VA, 22314, US

(21) Appl. No.: 10/522,377

(22) PCT Filed: Jul. 25, 2003
(86) PCT No.: PCT/GB03/03268

§ 371 Date: Jan. 26, 2005

(30) Foreign Application Priority Data
Jul. 26, 2002 (GB) 0217333.4

(51) Int. Cl. C12Q 1/68; C12M 1/34

(52) U.S. Cl. 435/6; 435/287.2

(57) ABSTRACT

There is described a system for multiparameter analysis of analytes. The system comprises: 1) primary supports (1) with a largest dimension (3) of 500 μm or less suspended in use in a fluid solution; 2) each primary support (1) comprises an identification means (2) for identification thereof; 3) at least one primary analyte (12) is bound to each primary support (1); 4) a secondary analyte (12′) is suspended in use in the fluid solution; and 5) a measuring means (25) is arranged in communication with the fluid solution for monitoring interaction between the primary analyte (12) and secondary analyte (12′). The system is distinguished in that: 6) secondary supports (1′) with a largest dimension at the most the same size as the largest dimension (3) of the primary supports (1) are suspended in use in the fluid solution; 7) each secondary support (1′) comprises an identification means (2′) for identification thereof; 8) at least one secondary analyte (12′) is bound to each of the secondary supports (1′); and 9) the measuring means (25) is arranged to detect any post-reaction interaction between one or more primary analyte (12) and one or more secondary analyte (12′) by detecting the identification means (2, 2′) of the primary and secondary supports (1, 1′) attached thereto. There is also described a method of multiparameter analysis of analytes using the system.

### FIELD OF THE INVENTION

The present invention relates to systems for multiparameter analysis of analytes in solution; moreover, the invention also concerns a method of performing such multiparameter analysis of analytes in solution.

### BACKGROUND TO THE INVENTION

There are many industries in which there is a requirement to study hundreds or thousands of samples simultaneously. Using traditional manual techniques of serial testing, such study has proved to be very time-consuming and expensive. Multi-parameter screening has hence become an important tool for processes in which rapid testing of many samples is required. For example, recent advances in our understanding of the human genome have led to a huge increase in the number of many novel drug targets being identified. At the same time, breakthroughs in the automated synthesis of chemical compounds has led to the availability of substantial libraries of compounds to be screened for possible pharmaceutical activity at these novel targets. Taken together, such developments have created a growing demand for more rapid, inexpensive and less labour-intensive analysis methods for drug discovery and development. Further examples of industries where multiple testing has found applications are in diagnostics, proteomics, the food industry (e.g. for detecting veterinary drug residues in foods, for monitoring undesirable and dangerous pathogens, and for identity preservation), and the cosmetics industry (e.g. for providing alternatives to animal testing, for screening active ingredients and novel molecules).

The development of multiplexing technologies has improved high-throughout screening processes. There are two main strategies employed, namely either physically separating each molecule to a particular place on a microarray (e.g. labelled tubes or wells, high density arrays or microchips), or performing reactions on individually encoded microcarriers, each carrier having a particular molecule bound to its surface.

In the first strategy, it is the exact location (x,y-coordinate) on the microarray that allows for identification of a target/compound which is analysed at that place. This method of tracking a reaction is usually referred to as positional or spatial encoding. Many different microarray formats are available commercially, e.g. the GeneChip® from Affymetrix. Characteristics of molecules being analysed on such microarrays must often be known and isolated beforehand; such prior knowledge makes it a complicated and costly process to manufacture specific microarrays to customer requirements with short lead times. A further disadvantage of spotted microarrays is the poor quality of the spotted molecule on the microarray. This results in low reliability of test results thereby obtained from the arrays. Such low reliability has, in turn, resulted in extensive quality control requirements during manufacture of the microarrays and spot arrays, or even high redundancy of each molecule built into the microarray, to ensure the quality of spotting. With the number of tests on each microarray increasing, use of advanced miniaturisation is required. Miniaturisation has also been used to decrease the amount of reagents. In addition, many companies and research institutes use homebrew methods for producing microarrays. The numbers of tests that can be performed on these home brews are very limited and also have the drawbacks described above. The reading of these homebrews is time and labour intensive with respect to the number of data points read.

In the second strategy, namely the second method of microcarrier-based assays, there arises a need to label each of the microcarriers (also called supports) to allow for identification of the molecules bound to their surface. This method allows for greater customisation by mixing the uniquely encoded microcarriers in one reaction vessel and subjecting them to an assay simultaneously. Those microcarriers that show a favourable reaction between the attached molecule and the target analyte may then have their code read, thereby leading to the identity of the molecule that produced the favourable reaction. An example of such a technology is Luminex Corporation's xMAP™ technology. The xMap system has a limitation of 100 differently optically coded microcarriers.

When the number of tested molecules on individual microcarriers increases advanced fluid handling is required. If the same identification code of a microcarrier is used for different molecules in different experiments there are contamination risks during the preparation of the microcarrier with attached molecule. When the number of microcarriers in an experiment increases, the problem of spectral overlap adds further problems of false positives and poor data quality. Other examples of microcarrier technology employ multicolour encoding similar to the xMap system are, for example, Illumina's BeadArray™ system, and Quantum Dot Corporation's Q-dot™ nanocrystals. Alternative methods of identification coding microcarriers used in traditional assays are the use of radio frequency identification (RFID) technology, e.g. PharmaSeq's micro-transponders described in a granted U.S. Pat. No. 6,361,950, or barcode identification technology, e.g. SmartBead Technologies' UltraPlex™ system described in an international PCT patent application having a publication no. WO0016893. These new approaches of encoding microcarriers have improved the signal-to-noise ratio of detecting such microcarriers.

Another encoded microcarrier solution includes the use of programmable matrices with memories as described in IRORI's published international PCT application no. WO 96/36436. This recording device stores the information of what molecules and biological materials are linked to the matrix material of each programmable matrice. These matrices can be in solution in one vessel or each linked to a well of a microtitreplate. Several matrices can also be arranged in an array taking the form of a microarray. Other particle array solutions from Virtual Arrays Inc and University of Hertfordshire are described in the published international PCT applications no. WO 00/63419 and WO 02/37944 respectively. These particle based arrays allow greater customisation of the probes (molecules), which probes are attached to coded particle arrays and tested against a test sample, than the positional based microarray solutions. These particle array solutions do require much automation and robotics when the number of multiplexed probes on uniquely identified particle arrays becomes very large into the hundreds or even thousands range. All the particle based array solutions do also have problems with cross reactivity for certain analytes/molecules when the number of multiplexing increases. Reaction between the particle array labelled molecules and a target analyte is detected using established detection methods like fluorescence, luminescence or radioactive labels, which often have limited shelf life.

Another system used to detect a target agent in a biological sample, described in an international PCT patent having a publication no. WO0242498, comprises a bead assay system that is read on optical biodiscs. This technology includes magnetic capture beads and reporter beads. Both sets of beads are coated with probes, which are complementary to the target molecule sought in the biological sample. If the target agent is present in the sample, the reporter bead and the magnetic capture bead binds to it. The bead complex is then isolated using a magnetic field and loaded onto an optical disc which has a capture layer affixed thereto. The presence of the dual bead complex can be detected either electromagnetically or based on fluorescence. The combination of different sizes of magnetic beads and different types of fluorescent reporter beads allows different target agents to be detected simultaneously. This dual bead systems experience many of the drawbacks described for previous described microarray and microcarrier-based assay methods of analysing target analytes with labour intense methodology, spectral overlap etc. They also require advanced sorting and reading equipment increasing the cost of their systems.

### SUMMARY OF THE INVENTION

A first object of the invention is to provide an improved system for the analysis of multiparameter analytes.

A second object of the invention is to provide a system to test large numbers of multiple parameters simultaneously.

A third object of the invention is to improve the parallel testing throughput of currently used microcarrier-based assay systems.

According to a first aspect of the invention, there is provided a system as defined in the accompanying claim 1.

The system is of advantage in that it is capable of addressing at least one of the aforementioned objects of the invention.

The system is beneficial in that it is flexible and can also be used to complement and/or improve existing support-based and/or microarray technologies. As all the reactions in the system are tagged by the interaction of individual identifiable supports, the throughput previously achieved using support-based technology for tagging primary analytes with supports and testing against a fluorescent labelled secondary analyte (e.g. the target analyte) is efficiently improved. The possibility of being able to test many primary analytes against many secondary analytes drastically decreases the number of experiments, the amounts of reagents used and the increases the amount of multiparameters possible to analyse simultaneously. Such improvement also allows the use of adapted conventional reading means, but requires the detection of the interacting primary and secondary supports' identification means. The possibility of this multiparameter testing with interacting individually identifiable supports substantially improves the analysis of for example the interactions of proteins or other large number of molecules with a number of compounds. By using two different sets of identifiable supports the benefit of these individually labelled supports become even more apparent. This allows the analysis of binding characteristics of primary and seconday analytes, previously difficult to achieve. The system is hence not limited to being able to test high numbers of primary analytes against only a single or very few fluorescently labelled secondary analytes. This further allows System biology experiments previously only possible to be performed in silica to now be performed empirically.

In a preferred embodiment of the invention, the primary supports are in the form of microparticles decreasing the amount of reagents used for each simultaneous testing process.

In a further preferred embodiment of the invention, the secondary supports are the same size or smaller than the primary supports as this allows improved possibility of quantification measurements of the secondary analytes present in a sample.

In another preferred embodiment of the invention, the identification means comprises one or more distinguishing geometrical features, such as shape, size, barcode or dotcode, enabling identification of each support. This allows the use of well established identification standards such as for example barcodes which give good signal to noise ratio and decrease the risk of spectral overlap and false positives.

Other preferred embodiments of the invention, comprises the use of radio frequency identification transponders (RFID) or optical identification, such as fluorescence or colour coding. The RFID gives the advantage of very large numbers of codes can be used and does not require visual communication between the measuring means and the identifiable support. The use of optical coding on the supports allows for combinations of wavelengths or colours not possible with standard fluorescent markers, e.g. FITC labelled, and allows for using low cost labelled supports.

In a further embodiment of the invention, there is provided a solid substrate, which accommodates the liquid solution. This allows the use of the multiparameter analysis using interacting primary and secondary supports to be used together with existing microarray technology for the analysis of a three way interaction between analytes.

According to a second aspect of the invention, there is provided a method as defined in the accompanying claim 15.

The method is of advantage in that it is capable of addressing at least one of the aforementioned objects of the invention.

It will be appreciated that features of the invention can be combined in any combination without departing from the scope of the invention.

### DESCRIPTION OF THE DRAWINGS

Embodiments of the invention will now be described, by way of example only, with reference to the accompanying drawings wherein:

FIG. 1 is a plan view and a side view of a single support (microcarrier) comprising a sequential identification;

FIG. 2 is a schematic sectional side view of a single support (microcarrier) with analytes attached thereto;

FIG. 3 is a schematic diagram of a system for multiparameter analysis of analytes;

FIG. 4a, b are schematic diagrams of the interaction between a primary and a secondary support according to a preferred embodiment of the multiparameter analysis system;

FIG. 5a, b are schematic diagrams of the interaction between a primary and a secondary support according to an alternative embodiment of the multiparameter analysis system;

FIG. 6 is a schematic diagram of a system for multiparameter analysis of analytes comprising multiple supports and a fixed array or substrate;

FIG. 7a, b are schematic diagrams showing the experiment reaction between supports, and a fixed array or substrate;

FIG. 8 is a schematic diagram illustrating a planar-based reader for interrogating the system of FIG. 3; and

FIGS. 9a, 9b are schematic top views of a planar substrate illustrating examples of the measuring path taken by the planar-based reader of FIG. 8.

### DESCRIPTION OF EMBODIMENTS OF THE INVENTION

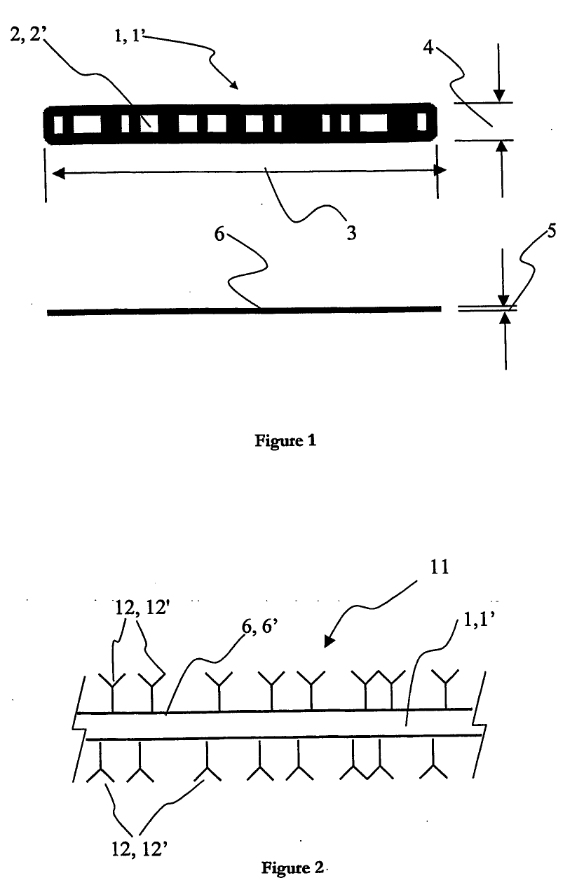

In FIG. 1, there is shown an illustration of a preferred embodiment of a support for use in a system according to the invention. This preferred embodiment of the support is susceptible to being used as a primary support 1 and/or a secondary support 1′ in the system for multiparameter analysis of analytes described further in the following detailed description. There is shown a single support 1, 1′; such a support will also be referred to as a microcarrier, microparticle or “bead” in the following description. The primary supports 1, 1′ can be fabricated from virtually any insoluble or solid material, for example one or more of polymers, silicates, glasses, fibres, metals or metal alloys. In the preferred embodiment of the invention, the supports 1, 1′ are fabricated from a metal, such as gold, silver, copper, nickel, zinc or most preferably aluminium. It is also preferable to use one or more polymers, such as polystyrenes, polyacrylates, polyamides, or polycarbonates when fabricating the supports 1, 1′. The support 1, 1′ is preferably either partially or totally coated in one or more of either of the above-mentioned materials.

The support 1, 1′ incorporates an identification feature 2, 2′ which is also referred to as an identification code or tag in the following description. Examples of the identification features 2, 2′ may be based on one or more of sequential identification, varied shape and size of the support, transponders (for example Radio Frequency Identification Chips, RFIs) attached to the support, and fluorescent coding or different colours of the support. Preferably, the identification feature 2, 2′ is a sequential identification which can be in the shape of at least one (or any combination thereof) of grooves, notches, depressions, protrusions, projections, and most preferably holes. The identification feature 2, 2′ being part of the support 1, 1′ is advantageous in that there is no need to label each support 1, 1′ after manufacture. The sequential identification 2, 2′ is suitably a transmission optical barcode, which is machine readable, allowing enhanced signal to noise ratio if read in transmission or even reflection. An associated sequential identification code is thereby recorded in the support 1, 1′ as a series of holes using coding schemes similar to those found on conventional bar code systems, for example as employed for labelling merchandise in commercial retailing outlets. Such a code allows the use of existing reader technology to determine the identification feature 2, 2′ of the supports 1, 1′, thereby decreasing the initial investment when adopting technology according to the invention.

In the preferred embodiment, the primary support 1 and/or secondary support 1′ is of substantially planar form with at least a principal surface 6 as illustrated in FIG. 1. The support 1, 1′ has suitably a width 4 to length 3 ratio in a range of circa 1:2 to circa 1:20, although a ratio range of circa 1:15 to circa 1:5 is especially preferred Moreover, the support 1, 1′ has a thickness 5 which is preferably less than circa 3 μm, and more preferably less than circa 1 μm. The thickness of less than circa 1 μm has been shown to provide sufficient mechanical support strength for rendering the support 1, 1′ useable in harsh experimental conditions. A preferred embodiment of the invention concerns the support 1, 1′ having a length 3 of circa 100 μm, a width 4 of circa 10 μm and a thickness 5 of circa 1 μm; such a support is capable of storing more than 100,000 different identification sequence bar codes 2. Experimental demonstrations of up to 100,000 different variants of the support 1, 1′ for use in bioassays for analyte characterization experiments have been undertaken. The current bar coding systems used have error and directional checking and up to 32 bits of information available on a support with a length of 100 μm. The support 1, 1′ is susceptible to being fabricated in various lengths 3 in a range of 40 μm to 100 μm, and carrying between two and five decimal digits of data in the sequential identification 2; examples of such a support 1, 1′ have been fabricated for use in different experiments for the detection of analyte characteristics.

Around 2.5 million supports similar to the support 1, namely primary and/or secondary supports 1, 1′, may be fabricated on a single 3-inch diameter semiconductor-type wafer, for example a silicon wafer, using contemporary established manufacturing techniques. Advantageously, the shape of the support 1, 1′ is such that it optimises the number of supports 1, 1′ manufactured per wafer and also substantially optimises the number of identification codes possible on the supports 1, 1′. Conventional photolithography and dry etching processes are examples of such manufacturing techniques used to manufacture and pattern a material layer to yield separate solid supports 1, 1′ with bar-coded identification 2, 2′.

A fabrication process for manufacturing a plurality of supports similar to the support 1, 1′ involves the following steps:
(1) depositing a soluble release layer onto a planar wafer;
(2) depositing a layer of support material onto the release layer remote from the wafer;
(3) defining support features, including the sequential identification 2, in the support material layer by way of photolithographic processes and etching processes;
(4) removing the release layer using an appropriate solvent to yield the supports released from the planar wafer; and
(5) optionally treating the support material to improve its attachment properties.

Many methods of chemically treating or physically altering the support material may be used for the optional step (5) to facilitate the attachment of an analyte, such as a test sample and/or a probe used in multiparameter experimental analysis, to the support 1, 1′. The treatment of the supports 1, 1′ can be performed after the release from the wafer as described above or alternatively prior to the release from the wafers or earlier in the manufacturing process steps. Alternatively, the treatment of the support material layer, step (5), could be omitted.

FIG. 2 provides an illustration of how analytes, namely primary or secondary analytes 12, 12′, are attached to a section 11 of the support 1, 1′. As described in the foregoing, the analytes 12, 12′ may be either probes or target analytes in test samples depending on how experiments utilizing the supports 1, 1′ are designed and customised. Different types of analytes 12, 12′ may be attached to supports 1, 1′ fabricated by steps (1) to (5) above, either before or after executing photolithographic operations or releasing the supports 1, 1′ from the planar wafer. By modifying the surface 6, 6′ of the supports 1, 1′ or the analytes 12, 12′, the attachment between analytes 12, 12′ and supports 1, 1′ is improved. Anodising the attachment surface 6, 6′ of the supports 1, 1′ is one way of providing such improved attachment enhancement. Aluminium is a preferred material for the supports 1, 1′ and there are known methods of growing porous surfaces through aluminium anodisation to improve the attachment properties thereof. Likewise, processes for sealing such porous surfaces are also known. The Applicant has exploited such knowledge to develop a relatively simple process for growing an absorbing surface having accurately controlled porosity and depth. Such porous surfaces 6, 6′ are capable of achieving a mechanical binding to preferred analyte 12, 12′. Using an avidin-biotin system is another approach for improving chemical binding between the supports 1, 1′ and their associated analytes 12, 12′. The supports' 1, 1′ surface 6, 6′ may also be treated with a binding material such as silane and/or biotin, to further enhance attachment properties. The supports 1, 1′ preferably have silane baked onto their surfaces 6, 6′. Attaching linking molecules, for example avidin-biotin sandwich system, to the analytes 12, 12′ further enhances their chemical molecular attachment properties.

The enhanced attachment is preferably achieved through having covalent bonds between attachment surface 6, 6′ of the support 1, 1′ and the analytes 12, 12′. The covalent bonds prevent the analytes 12, 12′ from being dislodged from the supports 1, 1′ and causing disturbing background noise during analysis. There is also a potential problem that loose analytes 12, 12′ could prevent the identification of reactions that have occurred. It is found to be important to wash the active supports 1, 1′, said supports 1, 1′ having analytes 12, 12′ attached thereto, after attachment to remove any excess analytes 12, 12′ that could otherwise increase the noise in the experiment during analysis. Discrimination of the tests is thereby enhanced through a better signal-to-noise ratio.

The primary support 1 and/or the secondary support 1′ described above utilises the benefits of a cost effective manufacturing technique with the possibility to tailor the design and identification coding as required. These benefits allows for the largest dimension 3 of the support 1, 1′ to be circa 500 μm or less, preferably circa 300 μm or less, more preferably circa 150 μm or less, most preferably circa 100 μm or less, circa 50 μm or less, or even circa 10 μm or less in length. By attaching a different primary analyte 12, 12′ to each support 1, 1′ with a specific identification code 2, 2′, a large number of analytes 12, 12′, such as molecules or other appropriate compounds, can be prepared for testing. As described in the foregoing, the shape as well as the size of the supports 1, 1′ may be varied as appropriate using microfabrication manufacturing techniques. Non-exhaustive examples of possible shapes are, for example, circular, elliptical, elongated, square, rectangular, multi-cornered or even multi-layered supports of the same or different materials. It is also, in some applications, preferable to have the primary supports 1 and/or the secondary supports 1′ in the size of nanoparticles with a largest dimension of circa 500 nm or less; examples of such nanoparticles comprise cylindrical nanobars. However, a lower limit to size is governed by sufficient sensitivity of the reaction kinetics being achieved.

In FIG. 3, a multiparameter analysis assay reaction 13 is depicted. The assay reaction 13 occurs in fluid solution according to a first embodiment of the invention. Preferably, the solution is a liquid which improves the sensitivity of the assay reaction. The assay 13 includes several suspended primary supports 1 covalently bound to primary analytes 12, such as target molecules. In the assay 13, there are also several suspended secondary supports 1′ covalently bound to secondary analytes 12′, such as test molecules. Many different target molecules 12 and test molecules 12′ are used for functioning as reaction molecules in an associated experiment to be performed, with each type of target molecule 12 being attached to a primary support 1 with a specific identification 2 and each type of test molecule 12′ being attached to a secondary support 1′ with specific identification 2′. If there is a match between one or more target molecule 12 and one or more test molecule 12′, they will mutually bind, preferably through a hydrogen bond, to generate a new dual support unit 16. The specific identification 2, 2′ on both the supports 1, 1′, can then be determined to indicate which target molecule(s) 12 and test molecule(s) 12′ have interacted. This system and methodology allows the analysis of multiple parameters in the same experiment. In the past, experiments have been limited to a very small number, often two to four, of fluorescent labelled target molecules. The simultaneous testing limitation has been due to the high risk of spectral overlap of the fluorescent labels. The utilisation of multiple supports 1, 1′ allows much greater numbers of multiplexing including multiple target molecules 12 and test molecules 12′.

By utilising the secondary supports 1′ instead of traditional reaction tags the benefits of being able to multiplex and customise the secondary analytes (target analytes) doubles compared to the traditional multiple particle based arrays tested against a single target analyte. With the identification of the secondary analyte 12′ through a secondary support 1′ a better reaction signal may be achieved. Also when the number of multiplexed analytes increases there are increasing problems with cross reactivity. In running a conventional particle array experiment cross reactivity between e.g. 1000 primary analytes verses 1 secondary analyte is more problematic than running an experiment using e.g. 100 primary analytes 12 on identifiable primary supports 1 verses 10 secondary analytes 12′ on identifiable secondary supports 1′. This allows very complicated biological systems with multiple dimensional features to be analysed through experiments rather than only in silica modelling.

An example of the methodology of performing these simultaneous testing according to this embodiment will now be described. The methodology involves a first step of providing several primary supports 1 with appropriately attached drug targets 12 suspended in a liquid solution. In a second step of the methodology, a suspension of several secondary supports 1′ with appropriately attached test compounds 12′ are added to the solution; such addition allows many different test compounds 12′ to be tested against many different target molecules 12 simultaneously, thereby dramatically improving testing throughput in comparison to contemporary methods. The resulting support units 16 are then detected by a measuring apparatus. In FIG. 3, it is also shown that the secondary supports 1′ are potentially smaller than the primary supports 1. Such a difference in size is capable of improving the reading of the primary supports' 1 identification 2, because the secondary supports 1′ do not interfere with, for example, a transmission barcode identification 2. There is also a reduction in the use of reagents as many more compounds are tested against many molecules simultaneously than what has been possible in the past. Preferably, each support 1, 1′ with a unique identification has only one type of analyte 12, 12′, e.g. a specific protein, attached thereon. It would however be possible to have more than one type of analyte 12, 12′ attached to each support 1, 1′ with a unique identification if multiple reactions were analysed on a support 1, 1′. As the analytes 12, 12′ are preferably attached to the supports 1, 1′ in solution the whole of the supports 1, 1′ are covered allowing good experimental sensitivity.

When performing a multiparameter analysis of analytes experiment, many different types of analytes 12, 12′ may be used. For the life science industry, the analytes 12, 12′ may be antibodies, antigens, proteins, enzyme substrate, carbohydrates, peptides, nucleic acids, peptide nucleic acids, cell lines, chemical components, oligonucleotides, serum components, drugs or any derivatives or fragments thereof. The multiparameter analysis system is very useful in the area of protein-protein interaction as there are very large numbers of proteins whose interaction needs to be investigated. For other industries, the analytes can be, for example, dyes, preservatives, labelling chemicals (for example for tracking the movement of counterfeit products), radioactive labelling chemicals, and food.

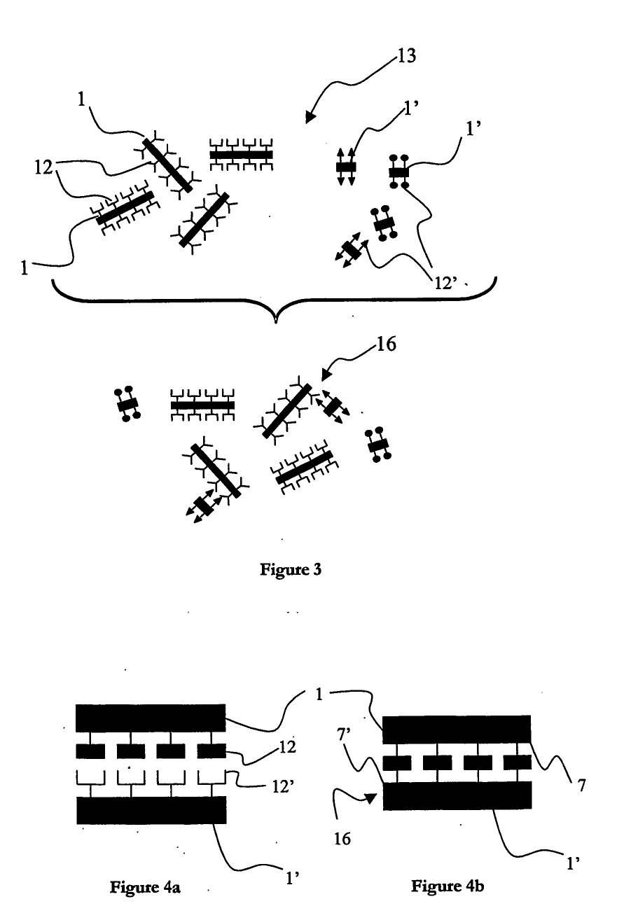

In FIGS. 4a and 4b, there is shown schematically the interaction of a matching pair of primary support 1 with bound primary analyte 12 and secondary support 1′ with bound secondary analyte 12′ pre- and post-reaction. These primary and secondary supports 1, 1′ each has bar-coded identifications 2, 2′ as described above. In this embodiment, the analytes 12, 12′ have been added to the supports prior to their release from their corresponding planar wafer during manufacturing, thereby resulting in the analytes 12, 12′ only being present on one side of the supports 1, 1′. It is also possible to achieve a similar effect by coating one surface of the supports 1, 1′ with a material that prevents the analytes 12, 12′ binding thereto. This coating further ensures that the primary and secondary supports only interact with a specific main surface 7, 7′ facing each other, thereby enabling the respective identification codes 2, 2′ to be read suitably in reflection or transmission with satisfactory signal-to-noise ratio. If circumstances arise where it would be desirable to separate the support units 16 from non reacted supports 1, 1′, a barrier sorting can be employed based on size or a sorting arrangement well known in the art of cell sorting is suitably used.

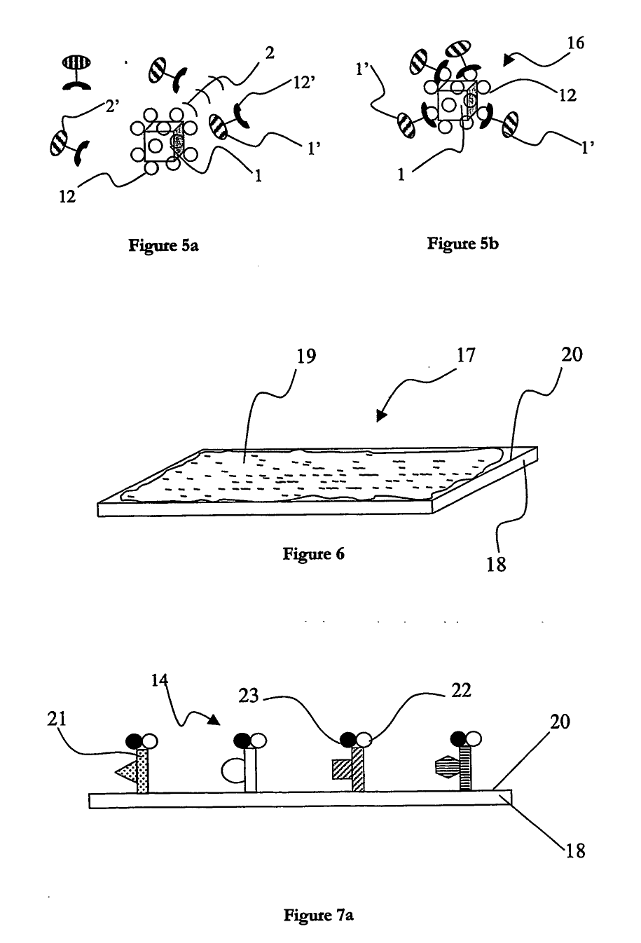

In FIGS. 5a and 5b, there is shown schematically an alternative embodiment of the multiparameter analysis system using supports 1, 1′ with different identification features 2, 2′. In this alternative embodiment, the primary support 1 is of cuboid shape and includes a radio frequency transponder identification (RWID) 2. The secondary support 1′ has a colour coded identification 2′. In FIG. 5b it can be seen how the primary analyte 12 on the primary support 1 binds directly to the secondary analyte 12′ on the secondary support 12′. The benefit of using a colour coded support rather than just a colour label as in traditional sandwich assays is that patterns or colour variations can be used to increase the codes possible, such as the Luminex xMap system. When the primary support 1 is made much larger than the secondary supports 1′, for example by at least a factor of 5, it becomes easier to measure quantitatively the amount of secondary analyte 12′ present in the sample provided that the amount of secondary analyte 12′ per secondary support 1′ is known. An alternative approach is to measure only yes/no interactions between the different supports 1, 1′. It is also preferable to use two different types of identification means 2, 2′ in the multiparameter analysis as there is less risk of the interference between the different identification signals.

The reader used for reading the supports 1, 1′ that have interacted to form a dual support units 16 is a modified version of the reader described in detail later in the detailed description with reference to FIG. 8. If the dual support units have different types of identification features 2, 2′ for the primary or secondary supports 1, 1′ a reading unit 30 capable of detecting two different identification signals must be used; the reading unit 30 is described later in FIG. 8. If the same type of identification means 2, 2′, such as barcodes, is used for the primary and secondary supports 1, 1′, the reader unit 30 needs to be adapted to allow reading of the both identification features simultaneously. For the embodiment described in FIGS. 4a and 4b, support 1, 1′ interrogation is potentially achievable by including two or more sub-readers in the reader unit 30 arranged mutually diagonally opposite to read the barcodes simultaneously in reflection or transmission. If large numbers of dual support units 16 are to be analysed, the thermodynamic principles of flow cytometry add the possibility of very high throughputs of up to several thousand units 16 per hour. Once again, the reader unit 30 is preferably then arranged to detect the supports' 1, 1′ identification features 2, 2′. The use of lasers for the reader unit 30 allows very small features of the support unit to be read, for example features of lateral size comparable to the wavelength of laser radiation employed.

In FIG. 6, there is shown schematically a system according to an additional embodiment of the multiparameter analysis system. The system is indicated generally by 17 and comprises a substrate (array) 18 accommodating a quantity of liquid solution 19 including supports 1, 1′. The substrate 18, which hereinafter also is referred to as an array or microarray, has two substantially planar main surfaces 20 and can be of any desired shape, but is more preferably rectangular, for example substantially square. The substrate 18 may also be made of a variety of materials, such as glass, metal, plastics materials, wafers, membranes or any other material contemporarily used for fabricating microarrays. Most preferably, the substrate 18 is fabricated from a material, for example glass (microscope slide) or plastics material (for example an acrylate), which is light transmissive. Such material characteristics potentially enable a support 1, 1′ with a transmissive bar-code identification feature 2, 2′ to be read in transmission whilst on the substrate 18. The substrate's 18 top main surface 20 is preferably planar or may be divided into sections by partitioning features, for example wells or boundaries, to prevent cross contamination between sections. The main surface 20 of the substrate 18 has preferably a surface area in a range of 0.25 cm² to 50 cm², more preferably in a range of circa 1 cm² to 25 cm² and most preferably in a range of circa 2 cm² to 6 cm². The liquid 19, which is placed on the substrate 18, is appropriately a liquid buffer solution and is normally an aqueous based solution. The system 17 can be considered to be an assay comprising the liquid solution 19 with loaded supports 1, 1′ placed on a substrate 18. The system 17 is of considerable advantage in that it is capable of providing the benefits of using two dimensional substrates 18 with established reader technology, multiplexing as well as the advantages of the multiparameter analysis system using microcarriers with higher throughput, good sensitivity and satisfactory reaction kinetics.

In a further embodiment of the invention, specific test molecules 12′ are attached to individual supports 1′ preferably through covalent bonds. Multiple test molecules 12′ can be tested for their affect on the activity of multiple target molecules 12 attached to individual supports 1 by placing the liquid solution 19 with suspended supports 1, 1′ on a substrate 18 with molecules 21 that are interrogating the target molecules 12 attached thereto. An example of the additional embodiment of the invention is employing a microarray pre-spotted with substrate molecules 21 to simultaneously test the activity of multiple test molecules 12′ attached to individual supports 1′ against multiple target molecules 12 attached to individual supports 1. A bonding reaction occurs when the molecules 21 do bind to the test molecule 12 and/or the target molecule 12′. The results of the reaction between test molecules 12′ and target molecules 12 will be based on the final position of the supports 1, 1′ together with their identification code 2, 2′.

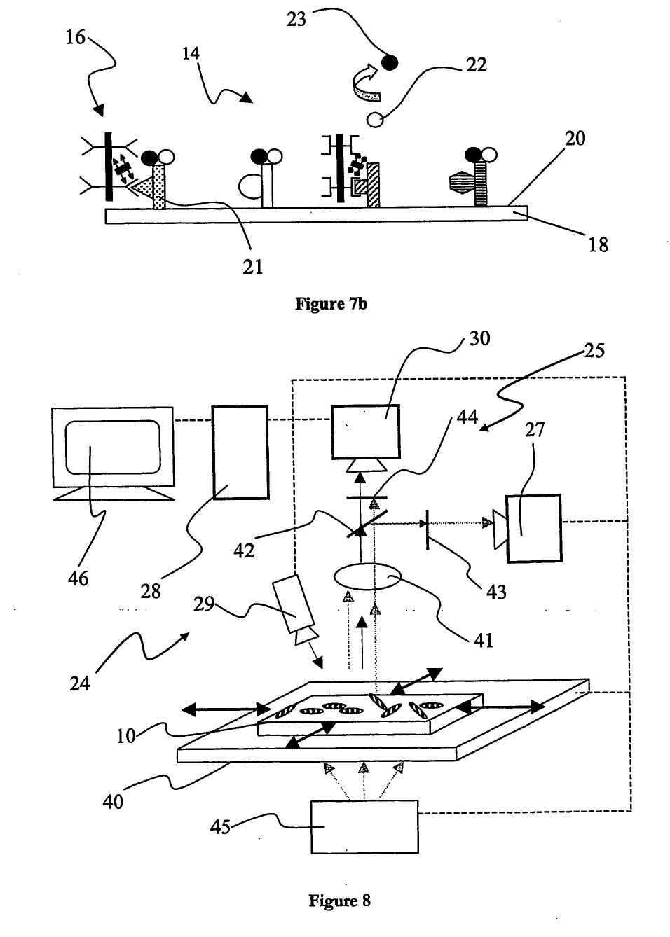

In FIGS. 7a and 7b, an assay reaction indicated generally by 14 is depicted which takes place on a substrate 18 according to the previous embodiment of the invention. The assay 14 consists of a liquid solution with suspended supports 1, 1′ and analytes 12, 12′. The analytes 12, 12′ are made up of target molecules 12 and test molecules 12′. Many different target molecules 12 and test molecules 12′ are used for functioning as reaction molecules in the experiment to be performed, with each type of target molecules 12 being attached to a primary support 1 with a specific identification 2, and each type of test molecules 12′ being attached to a secondary support 1′ with a specific identification 2′. The supports 1, 1′ preferably with at least one covalently bound target molecule 12 or test molecules 12′, thereon, are suspended in the liquid solution 19, which is then is placed on a main surface 20 of the substrate 18. The substrate 18 further has tertiary molecules 21, which act as substrates for the target molecules 12, 12′, bound to the main surface 20 through, e.g. a covalent bond. This potentially allows the use of a pre-spotted microarray as the substrate 18 in the system 17 to add another dimension to the multiparameter analysis. The molecules 21 bound to the substrate are suitably labelled with a fluorophore 22 and quencher molecule 23.

If there is a match between one or more target molecule(s) 12 and test molecule(s) 12′, they will mutually bind, preferably through a hydrogen bond, to generate a new dual support unit 16. If the test molecule 12′ inhibits the interaction of the target molecule 12 with its substrate tertiary molecule 21, the quencher molecule 23 will not be cleaved from the substrate molecule 21, thus the fluorophore 22 will remain quenched as shown in FIG. 7a. If, however, the test molecule 12′ binds to the target molecule 12, but does not inhibit the interaction of the target molecule with its substrate molecule 21, the quencher molecule will be cleaved and the fluorophore will emit a fluorescent signal when optically interrogated.

An example of this embodiment is the use of several supports 1 with appropriately attached enzyme targets suspended in a liquid solution. When performing an assay, a suspension of several supports with appropriately attached test compounds are added to the solution. Molecules that are known to be substrates for the enzymes would be pre-spotted onto the array substrate 18 at predefined positions. This allows many different test compounds to be tested against many different enzyme target molecules simultaneously to indicate not only whether or not the test compounds bind the target enzymes, but also the effect of said binding on the activity of the target enzymes.

Appropriate identification of supports 1, 1′, as mentioned above, refers to the importance of using a specific identification for a specific analyte 12, 12′, for example the target molecule 12 or the test molecule 12′. Such an arrangement also allows the use of predetermined identification codes 2, 2′ for certain analytes 12, 12′ but will also allow for matching of identification codes 2, 2′ and analytes 12, 12′ as desired when designing an experiment.

When performing tests of multiple target molecules 12 against multiple test molecules 12′, as in the described embodiments of the invention, it is also of benefit to analyse the experiments at different time points. This temporal analysis is potentially useful in pharmaceutical profiling where changes over time are important to record.

A reading system used for reading the substrate 18 with loaded supports 1, 1′ suspended thereon in a liquid solution 19 will now be described with reference to FIGS. 6, 7a and 7b.

Laser, ultra violet (UV) or light emitting diode (LED) reader equipment currently used for the analysis of, for example, microarrays or microcarrier-based assays is also susceptible to being employed with the aforementioned system for analysing multiple parameters of analytes 12, 12′. In the system, test results of reacting analytes 12, 12′ are measured as a yes/no binary result or by the degree of fluorescence emitted from a signal emitting label 23.

The system is indicated generally by 24 in FIG. 8 and comprises a reader. The reader includes a measuring unit indicated by 25 for measuring activity of the supports 1, 1′ tagged to analytes 12, 12′. The measuring unit-25 has a detection unit 27 to detect the fluorescent reaction signal from unquenched substrates 22 and a reader unit 30 to read the identification code 2, 2′ of the supports 1, 1′. The detection unit 27 has a fluorescence microscope for detecting the fluorescent signal indicating reaction. The reader unit 30 has a barcode reader to read the transmissive bar-codes 2, 2′ of the supports 1, 1′. It is preferable to have different type of signal for the support 1, 1′ identification 2, 2′ and the reaction detection, as there then is a limited risk of the signals being mixed up or being overlapping (spectral overlap). This allows for greater multiplexing (multiple simultaneous reactions) possibilities.

Once a sufficient number of supports 1, 1′ have been read, a processing unit 28 of the measuring unit 25 calculates the results of the tests associated with the supports 1, 1′. This sufficient number is preferably between 10 and 100 copies of each type of supports 1, 1′; this number is preferably to enable statistical analysis to be performed on test results. For example, statistical analysis such as mean calculation and standard deviation calculation can be executed for fluorescence associated with the unquenched fluorophores 22. A processing unit 28 is also included for controlling the detector and reader units 27, 30 so that the each individual support 1, 1′ is only analysed once.

Normally, all the supports 1, 1′ on the substrate 18 are analysed to verify the total quality of the experiment. In cases where there could be an interest in saving time and/or processing capacity, the software of the processing unit 28 can preferably be configured to analyse only the supports that have interacted and emit a signal, indicating that an interaction between characteristics of the analytes 12, 12′ has occurred. The analysis of the loaded substrate 18 using the measuring unit 25 is a very cost effective, easy to perform and suitable way to multiply the analysing capacity for low to medium sample numbers in the range of, for example, single figures to a few thousand supports 1, 1′ on each substrate 18.

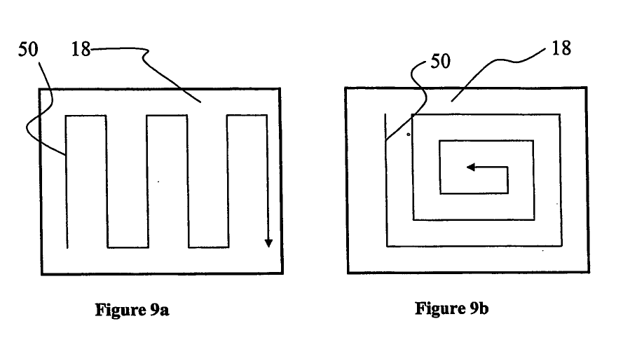

Preferred paths 50 for systematically interrogating the substrate 18 are shown in FIGS. 9a and 9b. FIG. 9a is a depiction of a meander-type interrogation regime, whereas FIG. 9b is a depiction of a spiral-type interrogation regime. There are of course many other possible paths 50 apparent to one skilled in the art, for example moving the substrate 18 in an opposite direction to the path 50, moving the substrate in a meandering diagonal path, or covering the whole substrate in one substantially linear path across its surface. However, the regimes of FIGS. 9a, 9b are efficient for achieving an enhanced support 1, 1′ read speed. A stepper-motor actuated base plate 40 supporting and bearing the substrate 18 may be used to move the substrate 18 around while the measuring unit 25 is held stationary. The most preferable method of analysis would, however, be to move the measuring unit 25 while the substrate is held stationary. The positions of supports 1, 1′ are tracked so that they are analysed once only.

The measuring unit's 25 reader unit 30 for image-processing is used to capture digital images of each field of the substrate 18 with a liquid solution 19 suspending supports 1, 1′ with attached analytes 12, 12′ thereon. Digital images thereby obtained correspond to light transmitted through the substrate 18 and past a base plate 40 and then through the supports 1, 1′ rendering the supports 1, 1′ in silhouette view; such silhouette images of the supports 1, 1′ are analysed by the reader unit 30 in combination with a processing unit 28. The sequential identification 2, 2′, for example a bar-code, associated with each support 1, 1′ is hence identified from its transmitted light profile by the reader unit 30. The signal emitting unit 29 generates a fluorescent signal, which signal makes the fluorophores 22 on the substrate molecules 21 fluoresce indicating a positive reaction.

The processing unit 28 is connected to the light source 45, the signal unit 29, the reader unit 30, and the detector unit 27 and to a display 46. Moreover, the processing unit 28 comprises a control system for controlling the light source 45 and the signal unit 29. The light silhouette and fluorescent signals from the fluorophores 22 on the substrate molecules 21 pass via an optical assembly 41, for example an assembly comprising one or more lenses and/or one or more mirrors, towards the detector unit 27 and reader unit 30. A mirror 42 is used to divide the optical signals into two paths and optical filters 43, 44 are used to filter out unwanted optical signals based on their wavelength. Alternatively, the light source 45 and signal unit 29 can be turned on and off at intervals, for example mutually alternately. Signals are received from the reader unit 30 and detector unit 27, which are processed and corresponding statistical analysis results presented on a display 46. Similar numbers of each type of supports 1, 1′ are required to give optimal statistical analysis of experiments. Such statistical analysis is well known in the art.

The intended uses of the system 17 may be in any process where experiments requiring the analysis of three dimensional multiparameter analysis of analytes. The applications where several parameters are involved are for example in biochemical detection of one or more analyte characteristics including, lead target identification and drug targeting. There will be many other applications for this system for alternative industries requiring multiparameter analysis of analytes.

Flow based reading of the experiments using primary and secondary supports 1, 1′ as identification of binding characteristics of the primary and secondary analytes 12,12′ provides an alternative or complement to the planar reader described previously and a fast and efficient way of analysing the reaction results. Sorting and detection of experiments using the primary and secondary supports 1, 1′ have been performed at a rate of ca 20 supports/second on the Union Biometrica, COPAS™, flow cytometer. The forward scatter and measurements of e.g. length and density give good indication of any interaction between the analytes 12, 12′ on the primary and the secondary supports 1, 1′. Sheath fluid in the flow cytometer focuses the supports 1, 1′ to the centre of a flow channel and allows detection using two lasers, which to cover the cross section the flow channel and arranged at ca 90 degrees to each other and with a joint focal pint at the centre of the channel. Both qualitative and quantitative results may be measured. Other flow readers which work well for analysing the multiparameter experiments using the embodiments of the primary and secondary supports 1, 1′ described are from DakoCytomation (MoFlo™) and Becton Dickenson (FACScan™).

It will be appreciated that modifications can be made to embodiments of the invention described in the foregoing without departing from the scope of the invention as defined by the appended claims. For example, when a conventional spotted microarray 18 or ELISA well plate with substrate molecules 21 attached directly to the array's 18 surface 20 is used as the array (substrate) 18 with positional identification in the system 14, the fluorophore 22 and quencher molecule 23 can be arranged to deactivate the fluorescent signal when a dual support unit 16 reacts with a suitable tertiary molecule 21.

1. A system for multiparameter analysis of analytes, the system comprising:
(a) primary supports with a largest dimension of 500 μm or less suspended in use in a fluid solution, wherein each primary support comprises identification means for identification thereof, and at least one primary analyte is bound to each primary support;
(b) a secondary analyte suspended in use in the fluid solution; and
(c) measuring means arranged in communication with the fluid solution for monitoring interaction between the primary analyte and secondary analyte, characterised in that:
(d) secondary supports with a largest dimension less than or equal to the largest dimension of the primary supports are suspended in use in the fluid solution, wherein each secondary support comprises identification means for identification thereof, and at least one secondary analyte is bound to each of the secondary supports; and
(e) the measuring means is arranged to detect any post-reaction interaction between one or more primary analytes and one or more secondary analytes by detecting the identification means of the primary and secondary supports attached thereto.

2. A system according to claim 1, wherein the largest dimension of the primary support is less than 300 μm.

3. A system according to claim 2, wherein the largest dimension of the primary support is less than 150 μm.

4. A system according to claim 3, wherein the largest dimension of the primary support is less than 50 μm.

5. A system according to claim 1, wherein the largest dimension of the secondary support is less than that of the primary support.

6. A system according to claim 5, wherein the largest dimension of the secondary support is less than 100 μm.

7. A system according to claim 6, wherein the largest dimension of the secondary support is less than 50 μm.

8. A system according to claim 7, wherein the largest dimension of the secondary support is less than 10 μm.

9. A system according to claim 1, wherein at least one of the identification means comprises one or more distinguishing geometrical features, such as shape, size, barcode or dotcode, enabling identification of each support.

10. A system according to claim 1, wherein at least one of the identification means is a radio frequency identification transponder (RFID).

11. A system according to claim 1, wherein at least one of the identification means is an optical identification, such as fluorescence or colour based.

12. A system according to claim 1, wherein the primary or secondary supports are only partially covered in their respective primary or secondary analyte.

13. A system according to claim 1, wherein the fluid solution is a liquid.

14. A system according to claim 13, wherein the liquid suspension is accommodated on a solid substrate, which substrate includes a main surface extending substantially in a two dimensional plane and has tertiary analytes fixedly arranged thereon for positional identification.

15. A method of multiparameter analysis of analytes, the method including the steps of:
(a) providing at least one primary support with a largest dimension of 500 μm or less and with identification means for identification thereof;
(b) binding at least one primary analyte to each primary support;
(c) suspending the primary support with its primary analyte and a secondary analyte in a fluid solution; and
(d) providing measuring means in communication with the fluid solution for monitoring interaction between the primary analyte and the secondary analyte, characterised in that the method further comprises the steps of:
(e) providing secondary supports with a largest dimension less than or equal to the largest dimension of the primary supports and with identification means for identification thereof;
(f) binding at least one secondary analyte to each of the secondary supports,
(g) suspending the secondary supports in use in the fluid solution, and
(h) arranging for the measuring means to detect any post-reaction interaction between one or more primary analytes and one or more secondary analytes by detecting the identification means of the primary and secondary supports attached thereto.

16. (canceled)

17. (canceled)
